(19) United States
(12) Patent Application Publication
(10) Pub. No.: US 2021/0154058 A1
(43) Pub. Date: May 27, 2021

(54) Absorbent extender tab insert

(71) Applicant: Dannielle Victoria Christiansen, Middletown, PA (US)

(72) Inventor: Dannielle Victoria Christiansen, Middletown, PA (US)

(21) Appl. No.: 16/974,242

(22) Filed: Nov. 23, 2020

Related U.S. Application Data
(60) Provisional application No. 62/939,938, filed on Nov. 25, 2019.

(51) Int. Cl. A61F 13/493; A61F 13/505; A61F 13/474; A61F 13/62

(52) U.S. Cl. CPC A61F 13/493; A61F 13/505; A61F 2013/15276; A61F 13/622; A61F 13/474

(57) ABSTRACT

An absorbent extender tab insert that may be used with a diaper or incontinent article via a nylon hook component. The absorbent extender tab insert prevents leakage of bodily fluids beyond the existing structure of the diaper or incontinent article. The absorbent extender tab insert can attach and re-attach as needed and is washable and reusable in certain embodiments. Moreover, the absorbent extender tab insert can be placed at the front of, back of or on side of diaper as needed to prevent leakage of bodily fluids.

### REFERENCE TO RELATED APPLICATIONS

This application makes reference to Provisional Application No. 62/939,938.

### FIELD

The present invention relates generally to diaper systems and flaps. More specifically, the present invention relates to inserts or tabs designed to prevent leakage of bodily fluids beyond the existing structure of standard diapers. The inserts or tabs attach to standard diapers with a one-sided nylon hook component feature. The present invention, an absorbent extender tab insert is directed to use with diapers and incontinent articles. In a similar embodiment, a reusable absorbent extender tab insert is disclosed which can be used with cloth diapers and washed and reused. The primary objective of the invention is to provide an extended absorbent adhesive, capable of preventing leakage of bodily fluids.

### BACKGROUND OF THE INVENTION

Various absorption articles are well known in the art and available commercially. Diaper systems have long been used with children as a way of containing urine and excrement prior to toilet training. Diapers generally contain absorbent material which prevents the soiling of outer garments. Diapers may also be utilized by adults or adolescents who are unable to use a toilet or who may be incontinent. Both disposable and reusable diapers alike are well known. See U.S. Pat. No. 5,409,476. The purpose of the present invention is to minimize the leakage of bodily fluids from these diapers or other incontinent articles. There is a need in the prior art for an extendable absorbent pad which can be placed at any point on these diapers or other articles with its novel nylon hook component and serve as a barrier against bodily fluid leakage.

Currently in the art, the use of gripping tabs attached to incontinent or sanitary articles is purposed. However, these gripping tabs fail to adequately absorb or prevent the leakage of bodily fluids. The primary function is in keeping the incontinent or sanitary napkin in place. Furthermore, they are generally manufactured as part of the article and each user is unable to place them in the position in which leakage commonly occurs. For example U.S. Pat. No. 5,755,710, discloses an absorbent article for use in the perineal area of the body. The article has an absorbent core which has an upper body facing surface, a lower garment facing surface, first and second longitudinal sides, and first and second transverse ends. A body fluid impervious sealing wrapper having upper and lower portions each having longitudinally and transversely extending edges along which the portions are joined is formed around the absorbent core so as to enclose at least the entirety of the garment facing surface, the longitudinal sides, and the transverse ends, thereby forming a seal. The article has a centrally disposed opening in the upper portion of the sealing wrapper thus forming a window on the body facing surface of the absorbent core that exposes it to fluid flow. There remains need in the prior art for an absorbent extender tab insert which can be used and re-used and which contains a novel nylon hook component which allows it to be placed as needed.

U.S. Pat. No. 4,072,150 discloses a diaper with a moisture absorbent insert strip to absorb moisture and resist premature spillover. It offers multiple layers of highly absorbent material overlaying a layer of moisture impervious material, with the insert arranged over the longitudinal central area of the main diaper so that when soiled or wet, it could be partially rolled over to cover the soiled or wet area and then removed without removing the main diaper, which would leave the main diaper clean and dry for further use. One distinct problem with this prior art is that the claimed insert strip only functions with the accompanying diaper. Other problems relate to the fact that the insert is not removable and cannot be placed in other locations, and it is not reusable.

U.S. Pat. No. 10,492,960 discloses a diaper insert with a liquid impermeable external surface, an inner surface, and an absorbency area adjacent to the inner surface. The diaper insert has a flap with an attachment end, a distal end, a midsection between the attachment end and the distal end, an inner surface, and an external surface. The attachment end is coupled to the outer surface. An absorbency area is adjacent to the inner surface and has at least one absorbent layer. The midsection has a width that is less than or equal to a width of the distal end. The diaper and flap are secured to the wear using side fasteners, where the front edge forms a seal with the wearer's abdominal area, and the rear edge forms a seal with the wearer's lower back. Likewise, this prior art accompanies the undergarment claimed and cannot be repositioned elsewhere.

U.S. Patent Application 2014/0188065 A1 discloses a contoured absorbent pad with a liquid permeable inner sheet, a liquid impermeable outer sheet, and an absorbent core located between the inner and outer sheets. The absorbent core is contoured in thickness to have an elevated area that defines two sides and a base edge. The two sides are joined at one end and diverge from each other at their opposite ends. The base edge is located between the divergent ends of the two sides. The liquid impermeable sheet further includes an adhesive band for securing the absorbent pad to the diaper. This adhesive band fails to correct the problem of adhesion failure due to moisture causing the absorbent pad to fail. Given that the purpose of the absorbent pad is to absorb fluids, this need has not been met.

What is lacking in the prior art is an absorbent extender tab insert with a nylon hook component enabling it to be used with standard diapers and incontinent articles. Additional features include its ability to be used in either the front or rear of a diaper or along a side. Additional features include the super absorbent polymer, the ability to be washed and reused in certain embodiments and the ability to be secured in place in a manner in which excess bodily fluids will not dislodge.

In another embodiment, the present invention provides a gusseted textile extender tab which is capable of being used, washed and reused with reusable cloth diapers. This gusseted textiled extender tab contains a nylon hook component to attach to reusable cloth diapers already in wide use.

### REFERENCES

| | | |
|---|---|---|
| 3,989,047 | Talivaldis Cepuritis | 1976 |
| 4,072,150 | Jacob Glassman | 1978 |
| 5,069,672 | Heather Wippler | 1991 |
| 5,360,422 | James R. Brownlee | 1994 |
| 5,409,476 | Fredrica Coates | 1995 |
| 7,805,768 | Christine Martz | 2010 |
| 7,819,851 | Birgitta Karlsson | 2010 |
| 8,048,054 | William L. Bunnelle | 2011 |
| 9,592,167 | Bert Bogaerts | 2017 |
| 10,016,318 | Jane Speak | 2018 |
| 10,231,887 | Theresa Knecht | 2019 |
| 10,492,960 | Ryan K. Delka | 2019 |
| 10,716,717 | John Lee Hammons | 2020 |
| 10,779,998 | Lin Miao | 2020 |
| 2014/0188065 A1 | Stephen Defrancesco | PUB. 2014 |

### BRIEF SUMMARY OF THE INVENTION

The primary objective of the invention is to provide an absorbent extender tab insert capable of attaching to disposable diapers and incontinent articles through the use of a nylon hook component. In certain embodiments, this invention is reusable and non-disposable for use with washable cloth or material non-disposable diapers or incontinent articles.

The presently disclosed invention relates to an absorbent extender tab which attaches to diapers and other incontinent articles with a nylon hook component. This absorbent extender tab can be placed using the nylon hook component feature in the most frequent location of bodily fluid leaks, for example at the rear of the diaper 330 or in the front of the diaper 340. This absorbent extender tab also can be utilized for adolescent or adult incontinent needs as it is capable of adhering to standard disposable diapers and cloth reusable diapers.

More specifically, the present invention relates to an absorbent extender insert designed to prevent leakage of bodily fluids beyond the existing structure of standard diapers.

The primary objective of the invention is to provide an extended absorbent which attaches to an article via a nylon hook component and is capable of preventing leakage of bodily fluids.

In one embodiment of this invention, the absorbent extender tab insert is made up of double-faced gusseted material which is washable and usable with washable cloth diapers.

### BRIEF DESCRIPTION OF THE DRAWINGS

FIG. 1 is a top view of the absorbent extender tab insert according to an embodiment of the invention. The nylon hook component is stitched 130 to the tab along the bottom linear side of the tab 140. Surrounding the curved portion of the tab, waterproof complex film 110 seals the super absorbent polymer 120 in place.

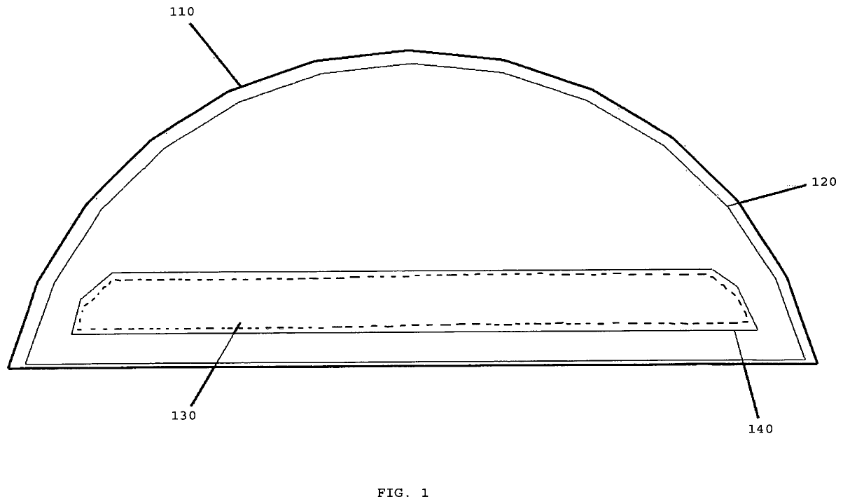

FIG. 2 is a top view of the reusable gusseted 220 material embodiment of this invention. The nylon hook component is stitched 250 to the tab along the bottom linear side of the tab 240. Surrounding the curved portion of the tab, double-faced material is stitched to seal in absorbent material 210. Each pocket 230 of gusseted stitching provides further absorbency for leaks.

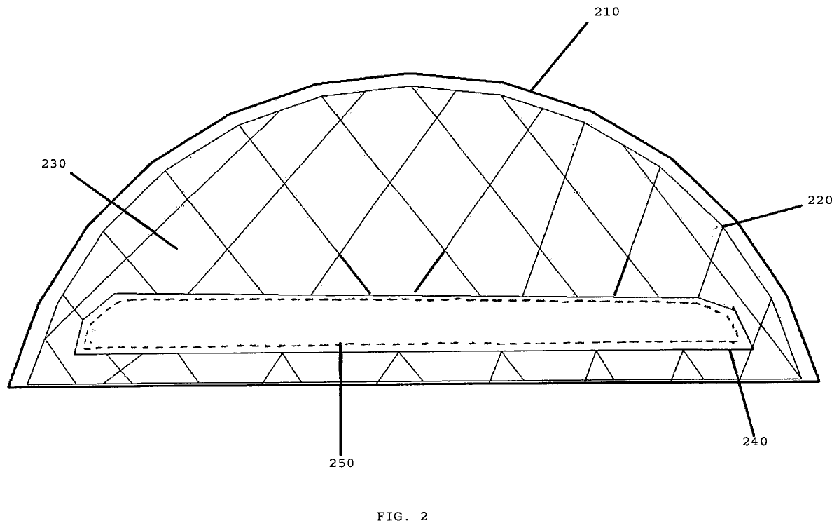

FIG. 3 is a perspective view of one embodiment of this invention, with the extender tab 310 connected to the back of the diaper 330. The front side 310 of the extender tab is made up of absorbent non-woven cloth and fluff pulp. The back side of this extender tab is sealed with waterproof complex film. The extender tab can be located at the front 340 of the diaper, or along the sides, wherever leakage is prone to occur.

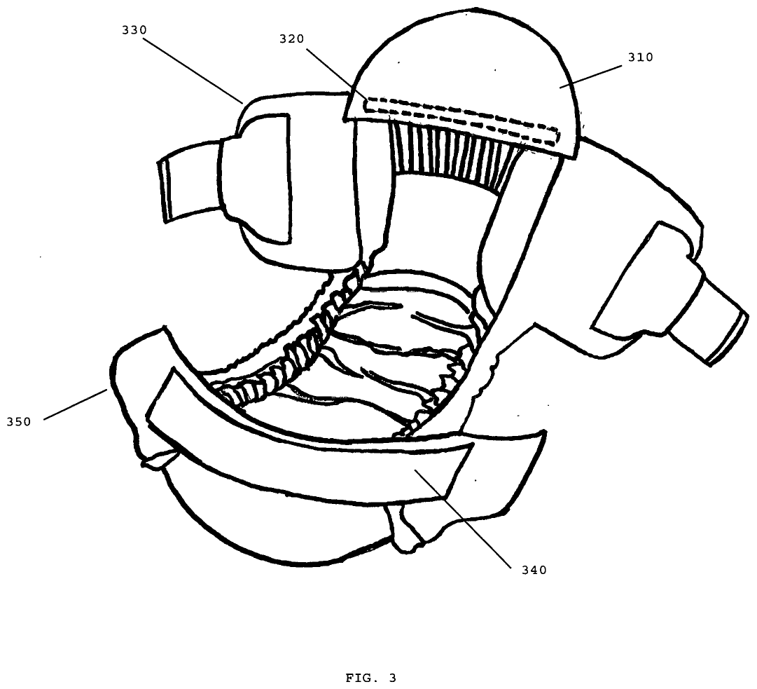

### DETAILED DESCRIPTION AND BEST MODE OF IMPLEMENTATION

The best mode of implementation is a curved absorbent extender tab insert which attaches to a diaper or incontinent article to absorb overflow of bodily fluids. The absorbent extender tab attaches by a strip of nylon hook component 140. Waterproof complex film, super absorbent polymer, fluff pulp, and non-woven cloth are layered together to produce a permeable absorbent side and a liquid impermeable barrier on the other side to prevent leakage of bodily fluids. A strip of nylon hook component is stitched onto the non-permeable non-absorbent side of the tab 130. This strip of nylon hook component attaches the tab to a diaper or incontinent article 320.

The present invention comprises a liquid permeable outer sheet on one side 310, a liquid impermeable outer sheet on a second side, and an absorbent core that is located between the two sheets. On the side with the impermeable waterproof complex film, a strip of nylon hook component is stitched to attach the absorbent extender tab to diapers 350 or incontinent articles as desired. The liquid permeable surface side is absorbent and may in some embodiments be gusseted to collect bodily fluids.

In some embodiments, for aesthetic purposes, the inner surface may have a pattern or other artistic decoration.

The present invention is reusable as long as the absorbent extender tab is not soiled. In order to reuse, simply pull the nylon hook component strip off the soiled diaper or incontinent article and reapply as desired. Once soiled, the absorbent extender tab can be removed and disposed of.

In another embodiment of this invention, the absorbent extender tab is reusable and washable. This embodiment is made up of double faced pre-quilted cotton or other gusseted material 220 and a strip of nylon hook component 240. Upon soiling, in this embodiment, the user simply pulls the nylon hook component apart from the standard diaper, washes the absorbent extender tab and re-uses.

What is claimed is:

1. A curved absorbent tab for use with a diaper or incontinent article, comprising:
a back side and a front side of the curved absorbent tab;
a super absorbent polymer substrate layer, a fluff pulp substrate layer, a non-woven cloth layer, and a waterproof complex film layer;
a strip of nylon hook component;
wherein the front side of the curved absorbent tab is the non-woven cloth layer, the back side of the curved absorbent tab is the waterproof complex film layer; and
the strip of nylon hook component is attached to the waterproof complex film that makes up the back side of the curved absorbent tab.

2. The curved absorbent tab identified in claim 1 wherein the back side, the front side, and the substrate layers are made of cloth, non-woven fabric, or other suitable material which is washable and reusable.

3. The curved absorbent tab identified in claim 1 wherein the back side, the front side, and the substrate layers are made of cloth, non-woven fabric, or other suitable material which is washable and reusable; and
the strip of nylon hook component is replaced with snaps, buttons, or other fastening connector means.
